(19) United States
(12) Patent Application Publication
(10) Pub. No.: US 2008/0306587 A1
(43) Pub. Date: Dec. 11, 2008

(54) Lens Material and Methods of Curing with UV Light

(76) Inventor: Jingjong Your, Cupertino, CA (US)

Correspondence Address: SHAY GLENN LLP, 2755 CAMPUS DRIVE, SUITE 210, SAN MATEO, CA, 94403, US

(21) Appl. No.: 12/177,720

(22) Filed: Jul. 22, 2008

Related U.S. Application Data
(63) Continuation-in-part of application No. 12/034,942, filed on Feb. 21, 2008.
(60) Provisional application No. 60/902,593, filed on Feb. 21, 2007.
(60) Provisional application No. 60/951,442, filed on Jul. 23, 2007.

(51) Int. Cl. A61F 2/16; C08F 2/46; C08F 20/10

(52) U.S. Cl. 623/6.11; 522/64; 522/182

(57) ABSTRACT

A polymeric material including a UV initiator, a UV absorber, and one or monomers, wherein the polymeric material is cured using UV light.

### CROSS-REFERENCE

This application is a continuation-in-part of application Ser. No. 12/034,942, filed Feb. 21, 2008; which claims the benefit of U.S. Provisional Application 60/902,593, filed Feb. 21, 2007, both of which are incorporated herein by reference in their entirety.

This application also claims the benefit of U.S. Provisional Application No. 60/951,442, filed Jul. 23, 2007, which is incorporated herein by reference in its entirety.

### BACKGROUND OF THE INVENTION

Two common types of polymerization initiators for ophthalmic device materials are thermal initiators and photoinitiators. Typical thermal initiators, including free radical initiators such as peroxides, initiate polymerization as temperature is increased. In some cases, two or three temperature tiers are involved such that curing involves a schedule of temperature/time combinations. Thermal initiation generally requires holding the monomer composition at elevated temperatures for lengthy periods of time. Total cure times of twenty-four hours are not unusual. See, for example, U.S. Pat. No. 5,290,892.

Photoinitiators generally offer the advantage of relatively short cure times and, unlike thermal initiators, can be used at ambient conditions, eliminating the need for high-temperature equipment or special ovens. Photoinitiators are activated by light of one or more specified wavelengths, rather than heat. Photoinitiation of ophthalmic lens materials is known. See, for example, U.S. Pat. No. 5,290,892.

One common type of photoinitiator known or used for curing ophthalmic lens polymers is UV-sensitive photoinitiators. UV-sensitive photoinitiators have not, however, been used with lens materials that contain a UV absorber and which are cured using UV light. UV absorbers present in an ophthalmic lens composition have been shown to interfere with the ability of UV-sensitive photoinitiators to efficiently cure the composition. UV absorbers are frequently incorporated in ophthalmic lens materials in order to reduce or block UV light from reaching the retina.

In addition to UV-sensitive photoinitiators, visible-light initiators are also known. U.S. Pat. No. 5,891,931 describes a curing process wherein the material is exposed to visible blue light.

There have also been efforts to incorporate a UV blocking chromophore into the initiator which results in a molecule with characteristics of both UV blocking and polymerization initiation. However, this approach limits the freedom of adjusting the amount of each components and the types of UV blocker in the formulation.

There remains a need for ophthalmic materials that include at least one UV absorber and at least one UV initiator that can be cured using UV light.

In addition, it may be desirable that the ophthalmic materials have an enhanced resistance to diffusion of fluids. It may also be desirable that the materials allow it to be deformed to a delivery configuration to enable its implantation in the eye, yet return to a pre-implantation configuration after being implanted in the eye. In addition, it may be desirable that the materials have a sufficiently high refractive index.

### SUMMARY OF THE INVENTION

One aspect of the invention is a method of preparing an ophthalmic material. The method includes preparing a mixture comprising a photoinitiator, a UV absorber, and at least one monomer, and exposing the mixture to UV light to sufficiently cure the mixture. In some embodiments the photoinitiator is a UV initiator. In some embodiments the UV initiator comprises a phosphine oxide group. In some embodiments the UV initiator is phenyl-bis-(2,4,6-trimethyl benzoyl)-phosphine oxide, 2,4,6-trimethyl benzoyl diphenyl phosphineoxide, Irgacure 2100™, or Irgacure® 2022, or a combination thereof.

In some embodiments the UV initiator is present in the amount of about 1% by volume.

In some embodiments the UV absorber is 2-(3-allyl-2-hydroxy-5-methylphenyl)-2H-benzotriazolem, which can be present in the amount of about 0.1% by volume.

One aspect of the invention is a method of preparing an ophthalmic material. The method includes preparing a mixture comprising a photoinitiator, a UV absorber, and at least one monomer, curing the mixture without using visible light. In some embodiments curing the mixture without using visible light comprises curing the mixture without using light with a wavelength above 400 nm. In some embodiments sufficiently curing the mixture comprises curing the mixture such that the % extractables in ethanol are less than about 6%.

One aspect of the invention is a polymeric material for an ophthalmic device. The material includes an alkyl acrylate, a fluoroacrylate, a phenyl acrylate, a photoinitiator; and a UV absorber, wherein the polymeric material is adapted to be cured with UV light. In some embodiments the photoinitiator is a UV initiator, which may comprise a phosphine oxide group. In some embodiments the UV initiator is phenyl-bis-(2,4,6-trimethyl benzoyl)-phosphine oxide, 2,4,6-trimethyl benzoyl diphenyl phosphineoxide, Irgacure 2100™, or Irgacure® 2022, or a combination thereof.

In some embodiments the UV initiator is present in the amount of about 1% by volume.

In some embodiments the UV absorber is 2-(3-allyl-2-hydroxy-5-methylphenyl)-2H-benzotriazole, which can be present in the amount of about 0.1% by volume.

In some embodiments the alkyl acrylate, which can be butyl acrylate, is present in the amount from about 35% to about 65% by volume.

In some embodiments the fluoroacrylate, which can be trifluoroethyl methacrylate, is present in the amount of about 15% to about 30% by volume.

In some embodiments the phenyl acrylate, which can be phenylethyl acrylate, is present in the amount of about 20% to about 40% by volume.

One aspect of the invention is a polymeric material for an ophthalmic device. The material includes a UV absorber present in the amount of less than about 1% by volume, a photoinitiator, and one or more monomers, wherein the polymeric material is adapted to be cured with UV light. In some embodiments the photoinitiator is a UV initiator, which can include a phosphine oxide group. In some embodiments the UV initiator is phenyl-bis-(2,4,6-trimethyl benzoyl)-phosphine oxide, 2,4,6-trimethyl benzoyl diphenyl phosphineoxide, Irgacure 2100™, Irgacure® 2022, or Irgacure® 819, or any combination thereof.

In some embodiments the photoinitiator is present in the amount of about 1% by volume.

In some embodiments the UV absorber is 2-(3-allyl-2-hydroxy-5-methylphenyl)-2H-benzotriazole, which can be present in the amount of about 0.1% by volume.

One aspect of the invention is an accommodating intraocular lens. The lens includes a light-transmissive optic portion and a peripheral non-optic portion extending from the optic portion, wherein the optic portion comprises a polymeric material comprising a UV absorber present in the amount of less than about 1% by volume, a photoinitiator, and one or more monomers, and wherein the polymeric material is adapted to be cured with UV light.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1-3 illustrate an exemplary accommodating IOL which can be made from one or more of the inventive polymeric compositions.

### DETAILED DESCRIPTION OF THE INVENTION

The invention relates to compositions of materials for ophthalmic devices. The compositions include a photoinitiator, a UV absorber, and at least one monomer. The photoinitiator is preferably a UV initiator and the compositions are cured using UV light. Although the composition includes a UV absorber, the composition is cured with UV light and yet can block harmful UV light from reaching the retina when the device is implanted in the eye.

While the polymeric materials can be used in a wide variety of applications, the polymers are described herein in their-use in an ophthalmic device such as an intraocular lens (“IOL”). While one use of the polymers is for a fluid-driven, accommodating IOL, the polymers can be used in a non-accommodating or non-fluid driven IOL. In addition to an IOL, the polymeric compositions of the present invention can also be used in other ophthalmic devices such as, but not limited to, contact lenses, keratoprostheses, capsular bag extension rings, corneal inlays, and corneal rings. An exemplary alternative use would be in the field of breast implants, such that the polymers can be used as an exterior shell-like material to prevent leakage of an internal material.

The compositions described herein may be used in any of the fluid-driven IOLs described in U.S. Provisional Application No. 60/433,046, filed Dec. 12, 2002; U.S. Pat. Nos. 7,122,053; 7,261,737; 7,247,168; and 7,217,288; U.S. patent application Ser. No. 11/642,388, filed Dec. 19, 2006; U.S. patent application Ser. No. 11/646,913, filed Dec. 27, 2006; and U.S. Provisional Application No. 60/951,441, filed Jul. 23, 2007; the disclosures of which are incorporated herein by reference in their entirety.

In preferred embodiments the initiator is a photoinitiator, and specifically a UV initiator—a photoinitiator that initiates the curing process in response to exposure to UV light.

In some embodiments the photoinitiator incorporates at least one phosphine oxide group. Such exemplary initiators include benzoylphosphine oxide initiators, which are known and are commercially available. Examples of benzoylphosphine initiators include 2,4,6-trimethylbenzoyl-diphenyl-phosphine oxide; bis-(2,6-dichlorobenzoyl)-4-N-propylphenylphosphine oxide; and bis-(2,6-dichlorobenzoyl)-4-N-butylphenylphosphine oxide.

In some embodiments Irgacure® 2022 is used as the UV initiator. Irgacure® 2022 is a mixture of Irgacure® 819 (20 wt %)+Darocur® 1173 (80% wt). The chemical name of Irgacure® 819 is phenyl bis(2,4,6-trimethyl benzoyl)phosphine oxide, and the chemical name of Darocur® 1173 is 2-Hydroxy-2-methyl-1-phenyl-1-propanone. In some embodiments Irgacure® 2022 is present in the amount of about 0.5% to about 5% by weight. In some embodiments it is present in the amount of about 0.5% to about 2% by weight. In some embodiments it is present in the amount of about 1%.

In some embodiments the photoinitiator is Darocur® TPO, the chemical name for which is 2,4,6-trimethyl benzoyl diphenyl phosphine oxide. In some embodiments the 2,4,6-trimethyl benzoyl diphenyl phosphine oxide is present in the amount of about 0.5% to about 5% by volume. In some embodiments it is present in the amount of about 0.5% to about 2% by volume. In some embodiments it is present in the amount of about 1% by volume.

In some embodiments the photoinitiator is Irgacure® 2100, which also includes a phosphine oxide group. In some embodiments the Irgacure® 2100 is present in the amount of about 0.5% to about 5% by volume. In some embodiments it is present in the amount of about 0.5% to about 2% by volume. In some embodiments it is present in the amount of about 1% by volume.

In some embodiments the photoinitiator is Irgacure® 819. In some embodiments Irgacure® 819 is present in the amount of about 0.2% to about 5% by volume. In some embodiments it is present in the amount of about 0.25% to about 2% by volume. In some embodiments it is present in the amount of about 0.5% to about 1% by volume.

UV light as described herein includes UVA light which generally has wavelengths between about 320 and about 400 nm, UVB light which generally has wavelengths between about 290 nm and about 320 nm, and UVC light which generally has wavelengths between about 200 nm and about 290 nm. UV light as used herein includes UVA, UVB, or UVC light alone or in combination with other type of UV light. In some embodiments the UV light source emits light between about 350 and about 400.

An exemplary UV light bulb used is model number F10T8BLB, made by USHIO Inc., which emits UV light in the range of about 310 to about 400 nm with peaks at about 368 nm. An alternative exemplary bulb is the UV Hex (40 Die) 375 bulb from Norlux Corp. Other exemplary bulbs have peak wavelengths about 352 nm. BLB lamps have tubes made of special deep blue filter glass that absorbs nearly all the visible light but transmits ultraviolet, often making an external filter unnecessary. Additionally, formulations containing a UV initiator can be cured using Lesco Superspot MK II, which is a high intensity UV spot curing device. The UV light source can also be a solid state source such as an LED.

The compositions also include at least one UV absorber. These absorbers prevent or inhibit UV light from damaging the eye. The UV absorber in the composition can be any compound which absorbs light having a wavelength shorter than about 400 nm, but does not absorb any substantial amount of visible light, and which is compatible with the composition. The UV absorber is incorporated into the composition mixture and is entrapped in the polymer matrix when the monomer mixture is polymerized.

Suitable UV absorbers include without limitation substituted benzophenones, such as 2-hydroxybenzophenone, 2-(2-hydroxyphenyl)-benzotriazoles, substituted 2-hydroxybenzophenones disclosed in U.S. Pat. No. 4,304,895, the 2-hydroxy-5-acryloxyphenyl-2H-benzotriazoles disclosed in U.S. Pat. No. 4,528,311, 2-(3′-methallyl-2′-hydroxy-5′-methyl phenyl)benzotriazole, or allyl hydroxymethylphenyl benzotriazole.

In some embodiments the UV absorber, such as 2-(3-allyl-2-hydroxy-5-methylphenyl)-2H-benzotriazole, is added in the amount, by volume, of less than 1%. In some embodiments it is less than 0.5%, and in some embodiments it is about 0.1% by volume.

Alternative UV absorbers may include β-(4-benzotriazoyl-3-hydroxyphenoxy)ethyl acrylate, 4-(2-acryloyloxyethoxy)-2-hydroxybenzophenone, 4-methacryloyloxy-2-hydroxybenzophenone, 2-(2′-methacryloyloxy-5′-methylphenyl)benzotriazole, 2-(2′-hydroxy-5′-methacryloyloxyethylphenyl)-2H-benzotriazole, 2-[3′-tert-butyl-2′-hydroxy-5′-(3″-methacryloyloxypropyl)phenyl]-5-chloro-benzotriazole, 2-[3′-tert-butyl-5′-(3″-dimethylvinylsilylpropoxy)-2′-hydroxyphenyl]-5-m-ethoxybenzotriazole, 2-(3′-allyl-2′-hydroxy-5′-methylphenyl)benzotriazole, 2-[3′-tert-butyl-2′-hydroxy-5-(3″-methacryloyloxypropoxy)phenyl]-5-chloro-benzotriazole and 2-[3′-tert-butyl-2′-hydroxy-5′-(3″-methacyloyloxypropoxy)phenyl]-5-chloro-benzotriazole.

The UV curing can occur over a period of 3 hours followed by 1 hours of postcuring in a thermal oven at 90° C. Optional thermal postcuring can also be done for two hours at 80° C. In some embodiments the composition is sufficiently cured in about 30 minutes. In some embodiments, the curing can occur in less than 30 minutes, such as about 15 minutes. In some embodiment the composition can be cured in about 5 minutes, or even less than 5 minutes.

An additional advantage of the compositions including the UV initiators and UV absorbers described herein is that they can be used with a plurality of monomers to enable the polymeric composition to retain specific desired physical properties.

When the composition is used in an ophthalmic device implanted in the eye (such as an IOL implanted in the capsular bag), the device becomes exposed to the fluid in the eye. The fluid in the eye can, over time, diffuse through the device and have unintended and/or undesired effects on the physical characteristics of the device. For example, a polymeric IOL that is implanted in the capsular bag may suffer from the diffusion of eye fluid into the IOL's polymeric material. Attempts have been made to coat ophthalmic devices with barrier layers to prevent such diffusion, but these procedures can be costly and time consuming. In addition, if an ophthalmic device contains a chamber or channel within the device which contains a fluid (e.g., a fluid-driven IOL), there is a risk that that fluid can diffuse out of its fluid chamber and into the polymeric material. This results in a decrease in the amount of fluid that can be utilized by the IOL, as well as to possibly alter the physical characteristics of the polymeric material. Therefore, the polymers described herein can be used in ophthalmic devices to resist the diffusion of fluid into or out of the device.

For implantable devices that must be implanted through an incision in the sclera, it is generally desirable that the incision in the sclera be as small as possible while still being able to deform the device without damaging it. The device must also be able to reform to its initial configuration after delivery. The inventive polymers described herein can therefore be used in ophthalmic device that need to be deformed to be delivered through an incision, yet will return to their initial configuration once implanted in the eye.

Similarly, it may be desirable to increase the refractive index (“RI”) of the ophthalmic device to increase its refractive power. An increase in the RI of the polymer can allow the device to be thinner, yet maintain a desired power. This can also provide the device with a smaller delivery profile to reduce the size of the incision in the eye during implantation.

Improved properties of the polymers described herein include, without limitation, the modulus of elasticity, the index of refraction, the resistance to the diffusion of fluids, the responsiveness of the composition, mechanical strength, rigidity, wettability, and optical clarity. These properties are not necessarily mutually exclusive and the list is not intended to be exhaustive.

In one embodiment the polymer comprises a first monomer, a second monomer, and a third or more monomers. In a preferred embodiment, the composition comprises butyl acrylate, trifluoroethyl methacrylate, phenylethyl acrylate, and ethylene glycol dimethacrylate as a cross-linker. These monomers are not intended to be limiting and are provided by way of example.

To achieve the desired properties of the polymer described above, it is contemplated that particular monomers or other components may be selected to achieve specific properties, or that particular monomers and other components may be selected in combination to achieve specific properties.

Butyl acrylate, for example, a rubbery material, generally enhances the responsiveness of the polymeric material. Alternatives for butyl acrylate include alkyl acrylates and other monomers with suitable responsiveness properties. Alternatives for butyl acrylate which may demonstrate responsive properties include, without limitation, octyl acrylate, dodecyl methacrylate, n-hexyl acrylate, n-octyl methacrylate, n-butyl methacrylate, n-hexyl methacrylate, n-octyl methacrylate, 2-ethylhexyl acrylate, 2-ethylhexyl methacrylate, 2,2-dimethylpropyl acrylate, 2,2-dimethylpropyl methacrylate, trimethylcyclohexyl acrylate, trimethylcyclohexyl methacrylate, isobutyl acrylate, isobutyl methacrylate, isopentyl acrylate, isopentyl methacrylate, and mixtures thereof. In addition, alternatives for butyl acrylate may include a branched chain alkyl ester, e.g. 2-ethylhexyl acrylate, 2-ethylhexyl methacrylate, 2,2-dimethylpropyl acrylate, 2,2-dimethylpropyl methacrylate, trimethylcyclohexyl acrylate, trimethylcyclohexyl methacrylate, isobutyl acrylate, isobutyl methacrylate, isopentyl acrylate, isopentyl methacrylate and mixtures thereof.

In some embodiments butyl acrylate is present in the range from about 10% to about 80% by volume, and in some embodiments is present in the range from about 20% to about 70% by volume. In preferred embodiments butyl acrylate is present in the range from about 35% to about 65% by volume, and in more preferred embodiments from about 45% to about 65% by volume. All percentages recited herein are considered to be “by volume,” unless specifically stated otherwise.

In some embodiments the polymer has a modulus of elasticity ranging from about 0.1 to about 0.6 Mpa. In some embodiments the modulus is between about 0.1 to about 0.3 Mpa.

Trifluoroethyl methacrylate, or suitable alternatives, can be added to the polymeric material to enhance the polymer's resistance to the diffusion of fluids as described herein. Generally, using a monomer with more fluorine atoms will enhance the polymer's resistance to the diffusion of fluid.

While the ethyl group of trifluoroethyl can potentially bind up to 5 fluorine atoms, a large number of fluorine atoms can reduce the refractive index of the polymer. In some embodiments, therefore, trifluoroethyl methacrylate will provide a desired balance between the polymer's resistance to diffusion and the polymer's refractive index.

Fluorocarbon monomers can enhance the polymer's resistance to the diffusion of fluid and some can be used as substitutes for trifluoroethyl methacrylate. Alternatives for trifluoroethyl methacrylate include fluoroacrylates and other monomers with that provide that polymer with suitable resistance to diffusion properties. Alternatives for trifluoroethyl methacrylate include, without limitation, heptadecafluorodecyl acrylate, heptadecafluorodecyl methacrylate, hexafluorobutyl acrylate, hexafluorobutyl methacrylate, tetrafluoropropyl methacrylate, octafluoropentyl acrylate, octafluoropentyl methacrylate, dodecafluoropheptyl methacrylate, heptafluorobutyl acrylate, trifluoroethyl acrylate, hexafluoro-iso-propyl methacrylate, pentafluorophenyl acrylate, and pentafluorophenyl methacrylate.

In some embodiments trifluoroethyl methacrylate is present in the range from about 5% to about 70%, and in some embodiments it is present in the range from about 10% to about 50%. In preferred embodiments it is present in the range of about 15% to about 30%, and in more preferred embodiments it is present in the range of about 18% to about 22%.

Phenylethyl acrylate, or suitable alternatives, can be included in the polymeric composition to increase the refractive index of the polymer. Phenyl groups in general can increase the refractive index of the polymer. Alternatives for Phenylethyl acrylate include phenyl acrylates and other monomers with that provide that polymer with suitably high refractive index.

Other groups which can be used to increase the refractive index of the polymer include, without limitation, benzyl (benzoyl), carbazole-9-yl, tribromophenyl, chlorophenyl, and pentabromophenyl. Exemplary monomers that can be used as alternatives to phenylethyl acrylate include, without limitation, tribromophenyl acrylate, 2-(9H-Carazole-9-yl)ethyl methacrylate, 3-chlorostyrene, 4-chlorophenyl acrylate, benzyl acrylate, benzyl methacrylate, benzyl methacrylamide, n-vinyl-2-pyrrolidone, n-vinylcarbazole, pentabromophenyl acrylate, and pentabromophenyl methacrylate, phenylethyl methacrylate, 2-phenylpropyl acrylate, or 2-phenylpropyl methacrylate.

In some embodiments phenylethyl acrylate is present in the range from about 5% to about 60%, while in some embodiments it is present in the range of about 10% to about 50%. In preferred embodiments it is present in the range of about 20% to about 40%, and in more preferred embodiments it is present in the range of about 26% to about 34%.

In some embodiments the polymer has a refractive index of between about 1.44 to about 1.52. In some embodiments the refractive index is between about 1.47 and about 1.52. In some embodiments the refractive index is between about 1.47 and about 1.5.

In some embodiments the composition also includes a cross-linking agent, such as ethylene glycol dimethacrylate. Examples of suitable crosslinking agents include but are not limited to diacrylates and dimethacrylates of triethylene glycol, butylene glycol, neopentyl glycol, ethylene glycol, hexane-1,6-diol and thio-diethylene glycol, trimethylolpropane triacrylate, N,N′-dihydroxyethylene bisacrylamide, diallyl phthalate, triallyl cyanurate, divinylbenzene; ethylene glycol divinyl ether, N,N′-methylene-bis-(meth)acrylamide, sulfonated divinylbenzene, divinylsulfone, ethylene glycol diacrylate, 1,3-butanediol dimethacrylate, 1,6 hexanediol diacrylate, tetraethylene glycol dimethacrylate, trifunctional acrylates, trifunctional methacrylates, tetrafunctional acrylates, tetrafunctional methacrylates and mixtures thereof.

Cross-linking agents may be present in amounts less than about 10%, less than about 5%, less than about 2%, or less than about 1%. The cross-linking agent(s) can cause the polymers to become interlaced within a tri-dimensional space, providing for a compact molecular structure having an improved elastic memory, or responsiveness, over the non-crosslinked composition. As described above, the compositions described herein may be used in variety of ophthalmic device, such as intraocular lenses.

The diffusion resistant properties of the inventive polymers described herein may be further enhanced by providing a barrier layer on the exterior surface of the ophthalmic device. In addition, if the device comprises a fluid chamber disposed within the device (such as a fluid chamber disposed in a fluid-driven accommodating IOL), the device can also have a barrier layer on the inner surface of the fluid chamber to increase the resistance to diffusing out of the fluid chamber. The barrier layer can be a thin layer of a fluorocarbon materials or polymers, examples of which include hexafluoroethane, hexafluoropropylene, hexafluoropropane, octofluoropropane, polytetrafluoroethylene, and 1H,1H,2H-perfluoro-1-dodecene. The barrier layer can be deposited or covalently bonded on the solid surfaces of the ophthalmic device, either individually or in combination through a variety of manufacturing processes. One common manufacturing process is plasma deposition.

The layers formed by plasma deposition will generally be very thin, for example, from about 20 to about 100 nanometers. Because fluorocarbon polymers generally have low refraction indices, a barrier layer with a thickness that is less than a quarter of the wavelength of visible light will not be seen with the naked eye.

As stated above, the polymers described herein may be used in an IOL with fluid disposed therein, such as in fluid chambers. In general, the viscosity of a fluid is related to the diffusion properties of the fluid; a low viscosity fluid can more easily diffuse through the polymer.

An ophthalmic device may contain silicone oil. The amount of silicone oil that diffuses through the polymer can be reduced by selecting a silicone oil with narrow molecular weight distribution, in particular with the removal of low molecular weight silicone oil molecules. A sequence of stripping processes is commonly used to remove low molecular weight components in silicone oil. In general, low molecular weight components will diffuse faster than higher molecular components. However, higher molecular weight components contribute to an increase in the viscosity which requires a greater force to drive the fluid throughout the IOL. Therefore, silicone oil with a narrow molecular weight distribution is preferred. The fluid disposed within the ophthalmic device is not limited to silicone oil and can be, for example, a saline solution.

In some embodiments, however, the IOL components are substantially index matched, such that the deflection of one of the surfaces of the IOL contributes significantly to any change in power during accommodation. For example, the bulk polymer will be substantially indexed matched to any fluid within the IOL. Substantially index-matched, as that phrase is used herein, include minimal differences in refractive indexes between components of the IOL. For example, if adhesives are used in the manufacturing of an IOL, those adhesives may have different refractive indexes but those differences will be negligible when considering the overall power changes of the accommodating IOL.

In some embodiments the T_{G} of the polymer is about −20° C., and can stretch to about 4× the length without breaking.

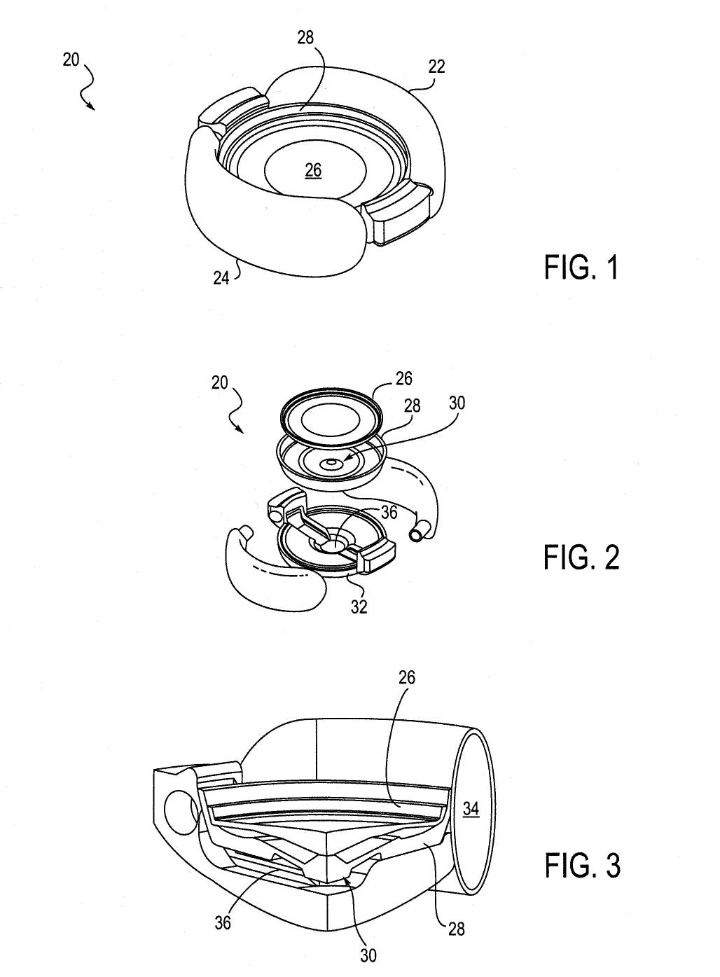

FIGS. 1-3 illustrate an exemplary embodiment of an accommodating IOL, at least part of which may comprise a polymeric composition described herein. IOL 20 includes a peripheral non-optic portion comprising haptics 22 and 24. IOL 20 also includes an optic portion comprising anterior element 26, intermediate layer 28, and substrate, or posterior element, 32. Intermediate layer 28 includes actuator 30. Haptics 22 and 24 define interior volumes 34 which are in fluid communication with active channel 36 defined by posterior element 32 and intermediate layer 28. The haptics engage the capsular bag such that zonule relaxation and tightening causes deformation of the haptics, which distributes a fluid disposed in the haptics and active channel between the haptics and the active channel. When fluid is directed from the haptics to the active channel, the pressure increase in the active channel deflects actuator 30, which deflects anterior element 26. This increases the power of the IOL.

Light entering the eye passes through the optic portion of the IOL. It may therefore be advantageous that the optic portion components comprise a UV absorber to prevent harmful UV light from reaching the retina. One or more optic portion components can therefore be made of a polymer which comprises a UV initiator, a UV absorber, one or more monomers, and is cured using UV light. The peripheral non-optic portion of the IOL, because it may not be in the path of light entering the eye, does not necessarily need to be made of a polymer including both a UV initiator and UV absorber. For example, it can be made from a polymeric composition which does not include a UV absorber, but includes a UV initiator and is cured with UV light. Sample #165 in Table 1 below is an example of such a polymeric composition.

### EXAMPLES

The following mixtures shown in Table 1 below were prepared. All of the compositions reached satisfactory polymerization under UV light from an USHIO F10T8BLB lamp at 368 nm. By comparison, the curing process was incomplete with other UV initiators, such as Darocur 1173. An optional step was thermal post-curing in an oven at 90° C.

TABLE 1

| | TEM | BA | PEA | EGDMA | Irgacure ® | Irgacure ® | Irgacure ® | TBDPO | Darocur ® | APB |
|---|---|---|---|---|---|---|---|---|---|---|
| Sample | (mL) | (mL) | (mL) | (mL) | 819 (mg) | 2022 (mL) | 2100 (mL) | (mg) | TPO (mg) | (mg) |
| 136 | 4 | 10 | 6 | 0.2 | | 0.2 | | | | 20.0 |
| 144 | 4 | 10 | 6 | 0.2 | | | | 200 | | 20.0 |
| 145 | 4 | 10 | 6 | 0.2 | | | 0.2 | | | 20.0 |
| 156 | 4 | 10 | 6 | 0.2 | | | | | 200 | 20.0 |
| 159A | 4 | 10 | 6 | 0.2 | 200 | | | | | 20.0 |
| 159B | 4 | 10 | 6 | 0.2 | 100 | | | | | 20.0 |
| 159C | 4 | 10 | 6 | 0.2 | 50 | | | | | 20.0 |
| 165 | 4 | 10 | 6 | 0.2 | 100 | | | | | |

Monomers: TEM—trifluoroethyl methacrylate; BA—n-butyl acrylate; PEA—phenylethyl acrylate.
Crosslinker: EGDMA—ethylene glycol di-methacrylate.
UV Initiators: Irgacure ® 2022, Irgacure ® 2100, Irgacure ® 819, Darocur ® TPO—2,4,6-trimethyl benzoyl diphenyl phosphine oxide; TBDPO—trimethyl benzoyl diphenyl phosphine oxide
UV absorber: APB—2-(3-allyl-2-hydroxy-5-methylphenyl)-2H-benzotriazole.

The cured polymers were then extracted in 200 proof ethanol in soxhlet for 72 hours to quantify the degree of polymerization. The degree of polymerization was determined by measuring percent extractables, calculated as (initial weight−final weight)/(initial weight)×100. The percent extractables were all less than 6%, and the specific results for some of the samples from Table 1 are shown below in Table 2.

TABLE 2

| Sample | Modulus (MPascal) | % Extractables |
|---|---|---|
| 165 | 0.301 | 3.494 |
| 159B | 0.334 | 4.050 |
| 159C | 0.2823 | — |
| 136 | 0.352 | 3.876 |

While embodiments of the invention have been described in some detail and by way of exemplary illustrations, such illustration is for purposes of clarity of understanding only, and is not intended to be limiting. Still further, it should be understood that the invention is not limited to the embodiments that have been set forth for purposes of exemplification, but is to be defined only by a fair reading of claims that are appended to the patent application, including the full range of equivalency to which each element thereof is entitled.

What is claimed is:

1. A method of preparing an ophthalmic material, the method comprising:
preparing a mixture comprising a photoinitiator, a UV absorber, and at least one monomer; and
exposing the mixture to UV light to sufficiently cure the mixture.

2. The method of claim 1 wherein the photoinitiator is a UV initiator.

3. The method of claim 2 wherein the UV initiator comprises a phosphine oxide group.

4. The method of claim 3 wherein the UV initiator is selected from the group consisting of phenyl-bis-(2,4,6-trimethyl benzoyl)-phosphine oxide, 2,4,6-trimethyl benzoyl diphenyl phosphineoxide, Irgacure 2100™, and Irgacure® 2022.

5. The method of claim 4 wherein the UV initiator is present in the amount of about 1% by volume.

6. The method of claim 1 wherein the UV absorber is 2-(3-allyl-2-hydroxy-5-methylphenyl)-2H-benzotriazole.

7. The method of claim 6 wherein the 2-(3-allyl-2-hydroxy-5-methylphenyl)-2H-benzotriazole is present in the amount of about 0.1% by volume.

8. A method of preparing an ophthalmic material, the method comprising:
preparing a mixture comprising a photoinitiator, a UV absorber, and at least one monomer; and
curing the mixture without using visible light.

9. The method of claim 8 wherein curing the mixture without using visible light comprises curing the mixture without using light with a wavelength above 400 nm.

10. The method of claim 1 wherein sufficiently curing the mixture comprises curing the mixture such that the % extractables in ethanol are less than about 6%.

11. A polymeric material for an ophthalmic device, comprising:
an alkyl acrylate;
a fluoroacrylate;
a phenyl acrylate;
a photoinitiator; and
a UV absorber, wherein there is an effective amount of the photoinitiator to cure the polymeric material with UV light.

12. The polymeric material of claim 11 wherein the photoinitiator is a UV initiator.

13. The polymeric material of claim 12 wherein the UV initiator comprises a phosphine oxide group.

14. The polymeric material of claim 13 wherein the UV initiator is selected from the group consisting of phenyl-bis-(2,4,6-trimethyl benzoyl)-phosphine oxide, 2,4,6-trimethyl benzoyl diphenyl phosphineoxide, Irgacure 2100™, and Irgacure® 2022.

15. The polymeric material of claim 11 wherein the UV initiator is present in the amount of about 1% by volume.

16. The polymeric material of claim 11 wherein the UV absorber is 2-(3-allyl-2-hydroxy-5-methylphenyl)-2H-benzotriazole.

17. The polymeric material of claim 16 wherein the 2-(3-allyl-2-hydroxy-5-methylphenyl)-2H-benzotriazole is present in the amount of about 0.1% by volume.

18. The polymeric material of claim 11 wherein the alkyl acrylate is present in the amount from about 35% to about 65% by volume.

19. The polymeric material of claim 18 wherein the alkyl acrylate is butyl acrylate.

20. The polymeric material of claim 11 wherein the fluoroacrylate is present in the amount of about 15% to about 30% by volume.

21. The polymeric material of claim 20 wherein the fluoroacrylate is trifluoroethyl methacrylate.

22. The polymeric material of claim 11 wherein the phenyl acrylate is present in the amount of about 20% to about 40% by volume.

23. The polymeric material of claim 22 wherein the phenyl acrylate is phenylethyl acrylate.

24. A polymeric material for an ophthalmic device, comprising:
a UV absorber present in the amount of less than about 1% by volume;
a photoinitiator; and
one or more monomers, wherein the polymeric material is adapted to be cured with UV light.

25. The material of claim 24 wherein the photoinitiator is a UV initiator.

26. The material of claim 25 wherein the UV initiator comprises a phosphine oxide group.

27. The material of claim 26 wherein the UV initiator is selected from the group consisting of phenyl-bis-(2,4,6-trimethyl benzoyl)-phosphine oxide, 2,4,6-trimethyl benzoyl diphenyl phosphineoxide, Irgacure 2100™, Irgacure® 2022, and Irgacure® 819.

28. The material of claim 24 wherein the photoinitiator is present in the amount of about 1% by volume.

29. The material of claim 24 wherein the UV absorber is 2-(3-allyl-2-hydroxy-5-methylphenyl)-2H-benzotriazole.

30. The material of claim 29 wherein the 2-(3-allyl-2-hydroxy-5-methylphenyl)-2H-benzotriazole is present in the amount of about 0.1% by volume.

31. An accommodating intraocular lens, comprising:
a light-transmissive optic portion; and
a peripheral non-optic portion extending from the optic portion,
wherein the optic portion comprises a polymeric material comprising a UV absorber present in the amount of less than about 1% by volume, a photoinitiator, and one or more monomers, wherein the polymeric material is adapted to be cured with UV light.
